(19) United States
(12) Patent Application Publication
(10) Pub. No.: US 2011/0118640 A1
(43) Pub. Date: May 19, 2011

(54) Back support belt with audio component

(76) Inventor: Brad Pollack, East Brunswick, NJ (US)

(21) Appl. No.: 12/590,834

(22) Filed: Nov. 16, 2009

(51) Int. Cl. A61F 5/00

(52) U.S. Cl. 602/19

(57) ABSTRACT

The back support belt with audio component represents a device which provides versatile support and comfort to men, women and pregnant women by delivering audio sound to an in-utero infant. The device is comprised of three section, a standard front belt section, standard back belt section and massage back belt section. The front belt section has a pocket which allows audio components to be held in specific locations to provide audio sound to an in-utero infant. The massage back belt section has a pocket which allows massage pads which will provide additional support and comfort. There are multiple massage pad inserts to select from, allowing the user to choose the specific type of comfort and support delivered. The design allows the user to select which functions to utilize at any given time.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

Back problems represent a common problem which many people deal with on a constant basis. There are many sources of back pain including pregnancy, which is what brought about the need for back support belts and maternity belts. Back support belts come in a variety of designs that alleviate pressure and provide support to the back area. Traditional maternity belts resemble a basic back support belt to support the additional weight located in the stomach area of a pregnant woman. Audio maternity belts have been introduced for the purpose of providing sound to an in-utero infant. The audio maternity belt is manufactured using headphones or speakers connected to a band that wraps around the waist. The design of an audio maternity belt may or may not include attributes of a support belt. The disadvantages of existing back support and maternity belts involve the lack of non-bulky massaging and audio components, and inability to easily modify the comfort by inserting different massage pads. The Back Support Belt with Audio Component represents a unique product that can be used as an adjustable back support belt and maternity belt with a pocket to insert a massage pad for comfort and support. The strategically positioned audio components enable the belt to deliver audio sound to an in-utero infant.

### BRIEF SUMMARY OF THE INVENTION

The Back Support Belt with Audio Component contains a pocket for massage pad inserts and an audio component for use as a maternity belt. The invention has 3 sections, a standard front belt section, standard back, belt section and massage back belt section. The standard front belt section enables the user to insert headphones of numerous styles, along with an elastic pocket which will hold an audio device. The standard back belt section is comprised of an elastic strap that will provide light back support while holding the front belt section onto the stomach of a pregnant woman for the purpose of providing audio sounds to the in-utero infant. The massage back belt section consists of a pocket, with dimension that covers the entire lower back. The pocket is accessed along the top allowing the user to insert a massage pad. The massage pads include but are not limited to memory foam pads, hot and cold gel pads, bead pads, vibrating pads and heating pads. The front and back belt sections are connected by elastic straps with either velcro, clips or side release buckles. The connection between the front and back belt section can be permanent on one side, making the maternity belt a one piece belt that wraps around the body.

### BRIEF DESCRIPTION OF THE SEVERAL VIEWS OF THE DRAWING

For a complete understanding of this invention, reference should be had to the following detailed description taken in connection with the accompanying drawings, in which:

FIG. 1A illustrates an interior view of the standard front belt section which comprises of a pocket for storing audio components, pocket for holding an audio device and connecting components. The connection component 11 serves the purpose of connecting the front belt section to the back belt section and is located on each side of the front belt section. The connection component 11 can be made with either side release buckles, clips or velcro straps. The stitching 17 which is sewn along the interior wall of the pocket serves the purpose of holding the audio components and the wire in specific locations within the pocket of the front belt section in order to deliver optimal audio sound for an in-utero infant. The interior wire hole 15 will allow the wire from the audio components to connect to the audio device while the device is held in the pocket located on the exterior of the front belt section. The apparatus 19 located along the top of the front belt section pocket provides access to the pocket for the purpose of inserting audio components. The apparatus 19 can be manufactured using a zipper, buttons or snaps.

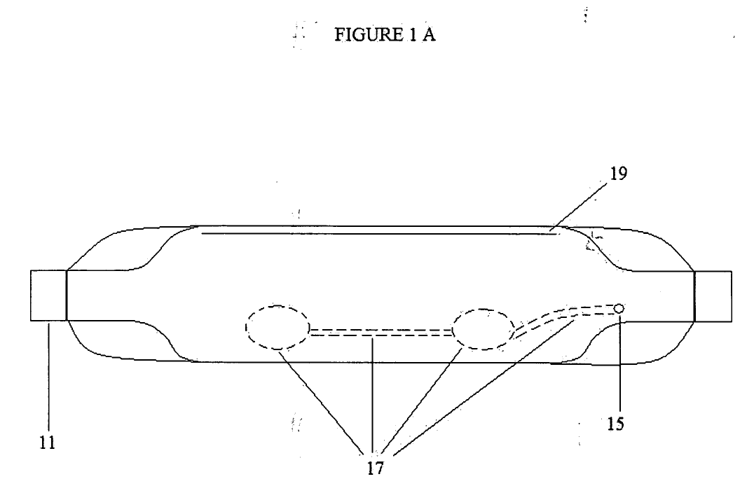

FIG. 1B illustrates an exterior view of the front belt section. The pocket 15 is made using a fabric in the shape of a pocket sewn onto the exterior of the front belt section. The fabric requires stretchy attributes to enable the storage of various audio device types and sizes. The exterior wire hole 17 enables the wire from the audio components stored in the pocket of the front belt section to be connected to the audio device held in the pocket 15.

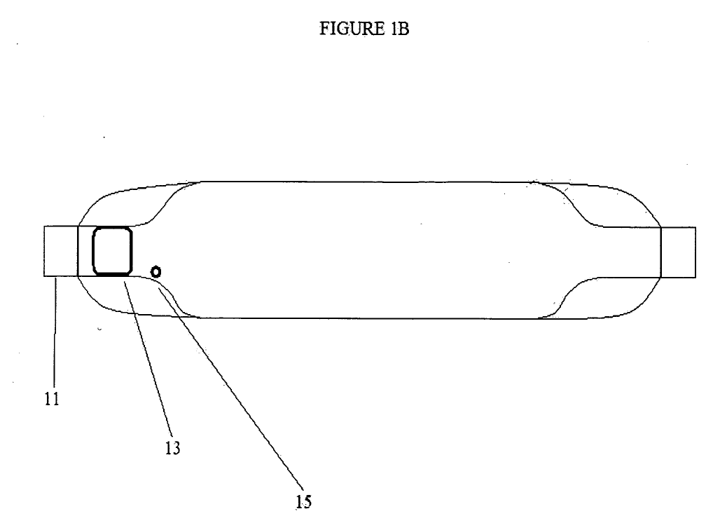

FIG. 2A illustrates the interior view of the massage back belt section. The connection component 10 serves the purpose of connecting the back belt section to the front belt section and is located on each side of the insertable back belt section. The connection component 10 can be made with either side release buckles, clips or velcro straps. The apparatus 14 located along the top of the massage back belt section pocket provides access to the pocket for the purpose of inserting massage pads. The apparatus 14 can be manufactured using a zipper, buttons or snaps. The stitching 16 which is sewn along the interior wall of the pocket serves the purpose of holding the massage pad in specific locations within the pocket of the massage back belt section in order to center the pad throughout the back area for optimal support and comfort.

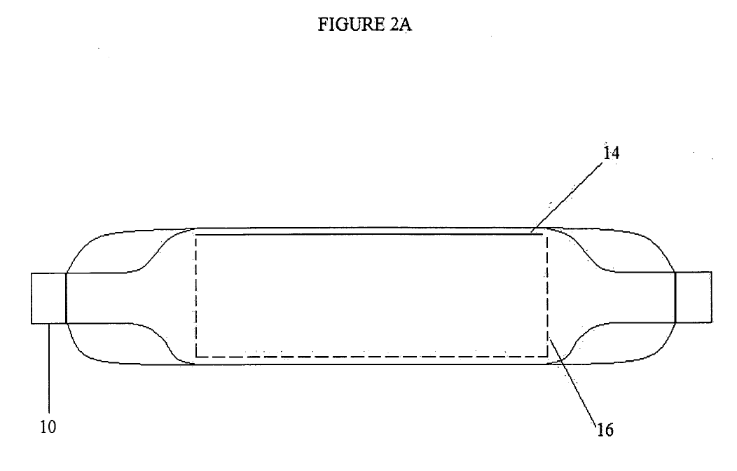

FIG. 2B illustrates the exterior view of the massage back belt section. The wire hole 12 provides access to plug in a massage pad that requires power into an electric outlet.

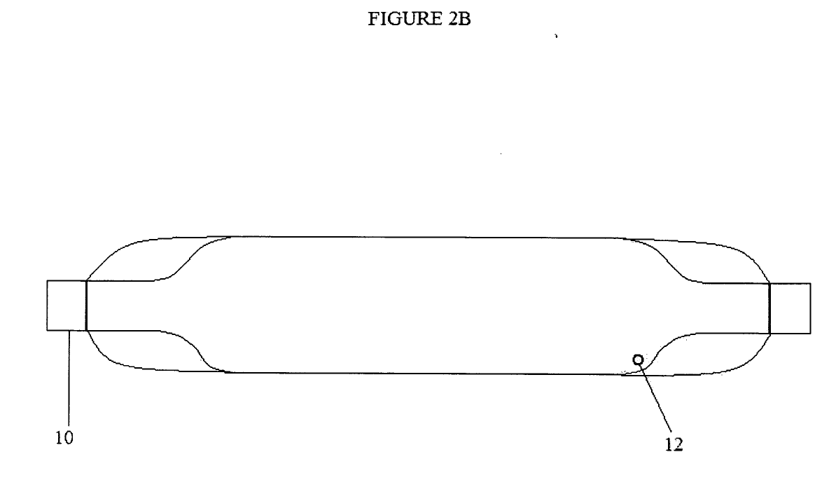

FIG. 3 illustrate the side view of a standard back belt strap. The standard back belt strap represents an elastic fabric with a connection component. The connection component serves the purpose of connecting the back belt section to the front belt section and is located on each side of the standard back belt section. The connection component can be made with either side release buckles, clips or velcro straps.

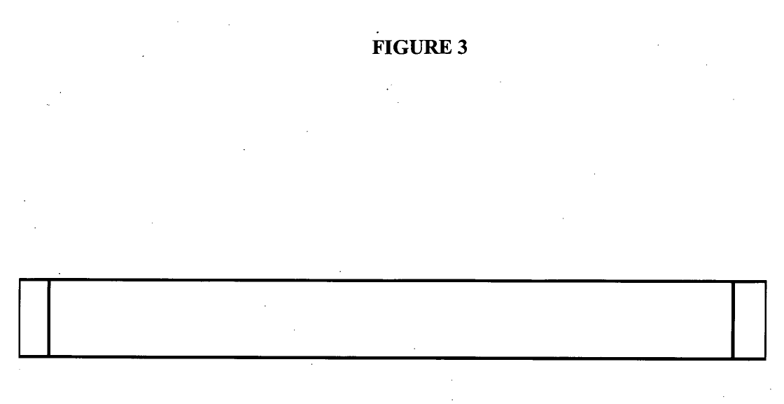

FIG. 4 illustrate an overhead view of the maternity belt comprising of the front belt section 1 and massage back belt section 2 connected through a connection component. The connection component can be permanent on one side allowing the invention to become a one piece belt that wraps around the body. This drawing illustrates the location of all components previously described in FIGS. 1A, 1B, 2A, and 2C.

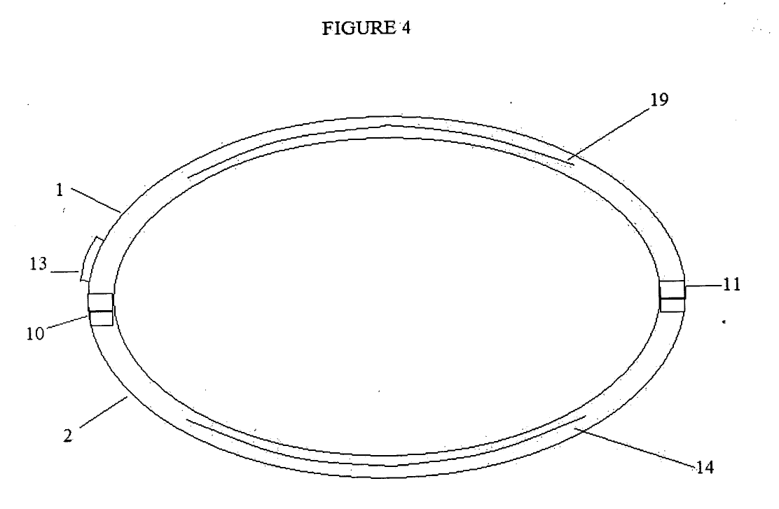

### DETAILED DESCRIPTION OF THE INVENTION

The Back Support Belt with Audio Component is comprised of three sections, the standard front belt section, standard back belt section and massage back belt section. The belt is created by combining the standard front belt section with one of the back belt section options, providing both comfort and support to the body, along with audio sound to an in-utero infant.

The front belt section consists of a fabric casing. The fabric composition requires a stretchy characteristic similar to that of nylon or spandex. The fabric may or may not have elastic straps embedded into the fabric to provide additional strength as an upgraded belt design. The casing includes a lower side and upper congruent side which is sewn together with thread around the edges in overlying opposing relation to said lower side. The casing may or may not incorporate padding for additional comfort, including but not limited to foam or gel. A zipper is located along the top of the standard front belt section creating an accessible pocket termed the main pocket. Additional sewing of the lower side to the upper congruent side create the sections within the main pocket which will hold the audio equipment and wires within a specific location of the main pocket. The main pocket is utilized to hold audio equipment providing sound to an in-utero infant. The main pocket will have a small hole in the corner to allow the wire from the audio component held in the main pocket to be accessed by an audio device. A device pocket made of a fabric composition similar to elastic will be sewn onto the exterior of the front belt section. This device pocket serves the purpose of holding an audio device to be connected to the audio components held in the main pocket of the front belt section. On each side of the front belt section casing are the connection components. These components comprise of elastic straps that may or may not be covered by a fabric. Sewn into the connection component is either velcro, clips or a side release buckle to serve the purpose of connecting the front belt section to the back belt section.

The standard back belt section is comprised of an elastic strap which may or may not be covered by a fabric casing for comfort purposes. Sewn into each side of the elastic strap is either velcro, clips or a side release buckle to serve the purpose of connecting the standard back belt section to the front belt section. When the standard front belt section is connected to a standard back belt section a maternity belt is created that serves the purpose of providing audio sound to an in-utero infant with back support.

The massage back belt section consists of a fabric casing. The fabric composition requires a stretchy characteristic similar to that of nylon or spandex. The fabric may or may not have elastic straps embedded into the fabric to provide additional strength as an upgraded belt design. The casing includes a lower side and upper congruent side which is sewn together with thread around the edges in overlying opposing relation to said lower side. The casing may or may not incorporate padding for additional comfort, including but not limited to foam or gel. A zipper is located along the top of the insertable back belt section creating an accessible pocket termed the main back pocket. Additional sewing of the lower side to the upper congruent side create the section within the main back pocket which will hold the massage pad in a specific location of the main back pocket. The main back pocket will have a small hole in the corner to allow access for the wire from the massage pad (when applicable) to be plugged into an electric outlet. On each side of the massage back belt section casing are the connection components. These components are elastic straps that may or may not be covered by a fabric. Sewn into the connection component is either velcro, clips or a side release buckle to serve the purpose of connecting the massage back belt section to the front belt section. When the front belt section is connected to a massage back belt section a support belt is created that serves the dual purpose of providing audio sound to an in-utero infant along with back support with massaging capabilities.

I claim:

1. For use in providing versatile back support and comfort to men and women, while delivering audio sound to an in-utero infant as a maternity belt, a back support belt with audio component comprising:
A standard front belt section made of a fabric casing with stretchy characteristics and connection components on each side.
A standard back belt section made of a fabric strap with stretchy characteristics and connection components on each side.
An massage back belt section made of a fabric casing with stretchy characteristics and connection components on each side.
Said front belt section serves the purpose of support as a belt while holding audio components which provide sound to an in-utero infant.
Said standard back belt section serves the purpose of securing the front belt section around the body to provide back support.
Said massage back belt section serves the purpose of securing the front belt section around the body while also providing versatile comfort using a pocket filled with a massage pad.
Said fabric casing may be manufactured using any woven natural or man-made fabric type. The casing including a lower side and upper congruent side is sewn together with thread around the edges in overlying opposing relation to said lower side. Additional sewing between the lower side and upper congruent side define the pockets located in the front belt section which holds audio components and the back belt section which holds the massage pads.
Said connection components are comprised of either velcro, clips or side release buckles sewn into an elastic strap which is sewn into the fabric casing, connecting a back belt section to the front belt section.

2. A back support belt with audio component as set forth in claim 1 wherein the front belt section main pocket will hold headphones in specific locations to provide optimal sound delivery to an in-utero infant. The pocket which is accessed by an apparatus has a hole to allow the headphone wire to be attached to any audio device which may be held in the device pocket located on the exterior of the front belt section.

3. A back support belt with audio component as set forth in claim 1 wherein the massage back belt section main back pocket will hold a pad which will provide additional support and comfort as a support belt. Massage pads include but are not limited to foam, gel, beads, vibrating or heating pads. The pocket which is accessed by an apparatus has a hole to allow a massage pad which requires power to run the wire from the massage pad to an electric outlet.
